(19) United States
(12) Patent Application Publication
(10) Pub. No.: US 2012/0172949 A1
(43) Pub. Date: Jul. 5, 2012

(54) SKIN RADIATION APPARATUS

(75) Inventors: Giovanna Wagenaar Cacciola, Eindhoven (NL); Siebe Tjerk De Zwart, Eindhoven (NL); Ingrid Maria Laurentia Cornelia Vogels, Eindhoven (NL); Tim Dekker, Eindhoven (NL)

(73) Assignee: KONINKLIJKE PHILIPS ELECTRONICS N.V., EINDHOVEN (NL)

(21) Appl. No.: 13/380,914

(22) PCT Filed: Jun. 21, 2010
(86) PCT No.: PCT/IB2010/052790

§ 371 Date: Mar. 9, 2012

(30) Foreign Application Priority Data
Jun. 26, 2009 (EP) 09163843.7

(51) Int. Cl. A61N 5/06

(52) U.S. Cl. 607/90

(57) ABSTRACT

A skin radiation apparatus (10) is described for providing a person's skin in a radiation area (12) of the apparatus with modulated photon radiation (14). The apparatus comprises a photon radiation source (18) for generating the photon radiation and a modulation facility (16) for causing a modulation of the total power density of photon radiation in the wavelength ranges from 300 to 700 nm, from 1900 to 2000 nm and from 2400 to 10.000 nm in the radiation area is modulated between a first and a second, mutually different level with a frequency of at least 0.1 Hz and of at most 10 Hz, wherein the first value is at least 20 mW/cm 2 and the second value is at most one fourth of the first value.

### TECHNICAL FIELD

The present invention relates to a skin radiation apparatus.

The present invention further relates to a method for providing a person's skin in a radiation area with photon radiation.

The present invention still further relates to a photon radiation profile.

### BACKGROUND ART

The main functions of the skin are to regulate body temperature and, more importantly, to protect our internal organs against the offenses of the outside environment. The skin is a protector against shock and damage to the body. The skin is composed of three functional layers: the epidermis, dermis and hypodermis or subcutis; each with its own unique functions.

The epidermis is the uppermost layer, usually comprised of 15-20 layers of cells. The epidermis continually undergoes the birth, life and death of cells which are created at the base of the epidermis and, after a two-week migration, are shed at the surface.

The dermis is made up of cells, which produce fibers (collagen and elastin), and houses the elastic support of the skin. Nerve endings located in the dermis function as receptors that detect changes in temperature and feel pressure, pain and vibration. Receptors for sensing warmth are present in this layer at a depth of about 0.3 to 0.6 mm from the surface of the skin.

Finally the subcutis functions as a cushion and as a storage site for reserve energy for the body.

Light treatment consists of exposure to daylight or to specific wavelengths of light using lasers, LEDs, fluorescent lamps, dichroic lamps or very bright, full-spectrum light, for a prescribed amount of time and, in some cases, at a specific time of day. It has proven effective in treating Acne vulgaris, seasonal affective disorder, neonatal jaundice, and is part of the standard treatment regimen for delayed sleep phase syndrome. It has recently been shown effective in non-seasonal depression. Demonstrable benefits are claimed of phototherapy with UVA and UVB radiation for skin conditions such as psoriasis. The principle of phototherapy was established in late 19th century by the Nobel laureate N. R. Finsen. He used light for curing skin disease. Development of light treatment is mainly ascribed to the introduction of laser therapy originally used in surgery.

Dependent on the wavelength range light absorption in the skin is mainly caused by melanin, hemoglobin and water. Melanin is a pigment produced by the melanocytes, cells which are present in the epidermis and in the hairs, which extend outside from the dermis. Haemoglobin is present in the blood in the blood vessels especially in the dermis. Water is substantially present in each of the functional layers of the skin. Generally speaking photon radiation in the UV and blue range is substantially absorbed by melanin and haemoglobin in the epidermis. At longer wavelengths the penetration depth of the radiation increases, probably influenced by the fact that the absorption of melanin and haemoglobin decreases (see FIGS. 1A and 1B). At wavelengths above 1500 nm however the absorption by water increases to a substantial value, contributing therewith to a decrease of the penetration depth for those wavelengths (see FIG. 1C).

### SUMMARY OF THE INVENTION

It is a purpose of the invention to provide a skin radiation apparatus having new application possibilities.

It is a further purpose of the invention to provide a method for providing a person's skin in a radiation area with photon radiation having new application possibilities.

It is a further purpose of the invention to provide a photon radiation profile having new application possibilities.

According to a first aspect of the present invention a skin radiation apparatus is provided. The skin radiation apparatus provides a person's skin in the radiation area of the apparatus with modulated photon radiation. The apparatus comprises a photon radiation source for generating the photon radiation and a modulation facility for causing a modulation of the total power density of photon radiation in the wavelength ranges from 300 to 700 nm, from 1900 to 2000 nm and from 2400 to 10.000 nm in at least a sub-area of the radiation area, between a first and a second, mutually different value with a frequency of at least 0.1 Hz and of at most 10 Hz. The magnitude of the first value of the power density is at least 20 mW/cm² and the magnitude of the second value is at most one fourth of the magnitude of the first value. The total power density is understood to be the power density integrated over the said wavelength ranges. Photon radiation having a power density of at least 20 mW/cm² is clearly sensed by the warmth receptors in the skin. Modulation of the power density of the photon radiation between the first and the second level with a frequency in the range of 0.1 Hz to 10 Hz results in the perception of a massage effect on the skin, provided that the skin sufficiently absorbs the photon radiation.

The radiation area is the area of the skin that may be irradiated by the photon radiation source when the apparatus is in a predetermined position and orientation with respect to the skin of the user. A said total power density modulation may then include a simultaneous modulation of the total power density in the entire radiation area, but this is not necessary the case. Alternatively the radiation area may be partitioned in sub-areas that are each associated with a respective photon radiation module of the radiation source, which photon radiation modules are individually modulated. Still alternatively a radiation beam of a radiation source may be swept over the skin surface within the radiation area, so that each time a different sub-area within the radiation area is irradiated. In any case the effect is that an area of the skin, which may be a sub-area of the radiation area, is provided with photon radiation for which the power density integrated over the specified wavelength ranges is modulated.

Photon radiation most suitable for achieving the massage effect has a wavelength in the ranges of 300 to 700 nm, 1900 to 2000 nm and 2400 to 10.000 nm. Photon radiation with a wavelength in these ranges is directly absorbed by the warmth receptors in the skin or it is absorbed by the epidermis, where the heat is rapidly conducted to the warmth receptors.

In particular the ranges 1900 to 2000 nm and 2400 to 10.000 nm are advantageous for use in an apparatus according to said first aspect of the invention, in that reflection of the skin for photon radiation having a wavelength in these ranges is relatively low, independently of the skin-type.

Particularly suitable are the ranges from 300 to 500 nm, 1900 to 2000 nm, 2400 to 2600 nm and 3600 to 4200 nm. Photon radiation in these wavelength ranges is substantially absorbed directly in the region of the skin comprising the warmth receptors. In particular the ranges 1900 to 2000 nm, 2400 to 2600 nm and 3600 to 4200 nm thereof are advantageous in that reflection of the skin for photon radiation having a wavelength in these ranges is relatively low, independently of the skin-type.

The specified power density is understood to be the power density of the photon radiation impingent on the skin. For some skin types a relative large fraction of the photon radiation may be reflected by the skin. In an embodiment therefore the first value is at least 50 mW/cm². In that embodiment also photon radiation in a wavelength range of 300 to 500 nm, is clearly perceived, also by persons having a skin type with a relatively high reflectivity for this radiation.

In an embodiment the first value for the power density is at most 200 mW/cm². A substantially higher value, e.g. a value higher than 500 mW/cm² implies a relative high power consumption, while it no longer contributes to a comfortable effect on a person.

Various photon radiations sources may be used, such as low pressure discharge lamps, light emitting diodes (LEDs), cluster discharge lamps, etc. Also some types of incandescent lamps may be used provided that they cool down sufficiently fast, such as incandescent lamps of type Reflect IR-P1N of ICX photonics. LEDs are however in particular advantageous as the power density of the emitted photon radiation can be accurately controlled as a function of time, and as they have a relatively high efficiency.

Also the modulation facility may be realized in various ways. In one embodiment the modulation facility is an actuator that causes a periodical movement of the photon radiation source, so that the generated photon radiation is projected to a moving sub-area within the radiation area. Alternatively the actuator may move an optical system, e.g. a mirror in a radiation path from the photon radiation source to the radiation area, instead of moving the photon radiation source itself. In again another embodiment an optical modulator, such as an optical shutter, e.g. an LCD device is arranged in the radiation path that is modulated in an open and a closed state. Therewith moving parts are avoided.

In a preferred embodiment the modulation facility includes modulation of the power supplied to the photon radiation source. This is advantageous in that moving parts are avoided and that the average power consumption of the device is low in comparison to methods where a modulation is applied after the photon radiation is generated. On the other hand an embodiment wherein a modulation is applied after the photon radiation is generated has the advantage that it is also possible to use a photon radiation source that cannot be rapidly modulated, e.g. high pressure discharge lamps and most incandescent lamps.

In the embodiment wherein the modulation facility modulates the power supplied to the photon radiation source a light emitting diode (LED) is particularly advantageous as the photon radiation source as its photon radiation output can be easily controlled. Nevertheless also certain types of incandescent lamps may be used as indicated above.

In an embodiment the radiation source comprises a plurality of radiation modules that are switched on during mutually different time intervals. The radiation source may for example comprise 10 radiation modules that each irradiate the skin in a respective sub-area of the radiation area. The respective sub-areas may be distinct or may partially overlap. Various geometrical arrangements may be possible, e.g. the radiation modules may form a set of concentric circles or a set of parallel strips. The radiation source may have a mode of operation wherein a radiation module is switched on when its predecessor is switched off When the last radiation module in a sequence is switched off the first radiation module is switched on again. Instead of switching on a radiation module, e.g. a strip, at the moment that its preceding radiation module, e.g. a preceding strip, is switched off, the time intervals during which the radiation modules are switched on may overlap. Alternatively some time may lapse between the point in time that a radiation module is switched off and the point in time that a next radiation module is switched on.

In another embodiment the setting of the levels for the power density of the photon radiation is controlled as a function of time. In an embodiment the magnitude of the first level is gradually increased in order to compensate for the adaptation of the sensitivity of the skin to the warmth sensation.

This is also applicable to the embodiment wherein the radiation source comprises a plurality of radiation modules that are switched on during mutually different time intervals. In said embodiment for example in a first cycle each consecutive radiation module may be powered at a higher level so that it provides the skin with a higher power density than its predecessor. In a second cycle, following the first cycle each consecutive radiation module may be driven with a lower power. This pattern may be repeated.

The modulated photon radiation that is directly sensed by the warmth receptors, e.g. radiation in the wavelength range of 300 to 700 nm, 1900 to 2000 nm and/or 2400 to 10.000 nm, hereinafter called primary radiation, may be combined with additional radiation, e.g. with photon radiation that has a therapeutical or another effect, provided that the primary radiation is sufficiently modulated to perceive the massage effect. If said additional radiation is in one of said wavelength ranges of primary radiation it may be modulated synchronously with the primary radiation to prevent that it inhibits the massage effect. I.e. the additional radiation is modulated synchronously with the modulation caused by the modulation facility. This is also advantageous as in that case the primary radiation and the additional radiation may be provided by the same photon radiation source.

In an embodiment the skin radiation apparatus comprises a facility for generating additional photon radiation having a wavelength in a range of 700 to 1600 nm. Photon radiation having a wavelength in this range, for example in a subrange of 800 to 1500 nm, e.g. photon radiation having a wavelength of 870 nm penetrates through the upper layers and directly warms the deeper layers of the skin without substantially triggering the warmth receptors in the upper layers of the skin. A very high penetration of the photon radiation is achieved for photon radiation with a wavelength in a range from 1100 to 1400 nm, e.g. having a wavelength of 1320 nm.

The additional radiation may be modulated synchronously with the primary radiation. Alternatively, however, as radiation having a wavelength in the range of 700 to 1600 nm is not perceived, at least not immediately, by the warmth receptors, it may be provided continuously without disturbing the massage effect of the primary radiation.

The modulated primary radiation may also be combined with other additional radiation. For example it has been found that a combination of radiation with a wavelength of 590 nm and radiation in the IR range tends to reduce wrinkles Other types of additional radiation are useful for the treatment of cellulites. Also the application of modulated primary radiation has been found useful for pain relief. For example depilation methods using photon radiation are known to be painful. By combining the photon radiation for depilation with the modulated primary radiation, the massage effect so achieved substantially relieves the discomfort of the depilation treatment.

In an embodiment the skin radiation apparatus has a timer for interrupting operation of the apparatus after a predetermined time. The predetermined time may be set by the user, e.g. within a range that is predefined by the manufacturer.

In an embodiment the skin radiation apparatus has a distance sensing facility for generating a distance signal indicative for a distance between the radiation source and the radiation area. The distance signal may be used to interrupt operation of the photon radiation source if the distance is estimated less than a threshold value, e.g. a safety related minimum operating distance. Alternatively the distance signal may be used to control the photon radiation source so that the first value of the power density in a radiation area proximate or on the skin is substantially independent of the distance between the photon radiation source and the radiation area. Alternatively the photon radiation source, e.g. a laser, such as a semiconductor laser, may generate substantially parallel photon radiation beams, so that inherently the power density is substantially independent of the distance to the photon radiation source.

In an embodiment the skin radiation apparatus is provided with an optical detection facility for providing an optical detection signal. The optical detection signal may indicate whether the users skin is present in the radiation area, and if so what the type of skin is. Dependent on the indications of the optical detection signal the operation of the apparatus may be controlled. E.g. the apparatus may be automatically brought into an operational state if a skin is present in the radiation area, and operation may be interrupted if this is not the case. Dependent on the type of skin detected a property of the photon radiation provided by the photon radiation source may be adapted. For example if it is detected that a white skin is present within the radiation area, the first value of the power density may be increased to compensate for the higher reflection of the skin.

The optical detection signal may further be indicative for the state of the skin. Operation of the skin radiation apparatus may be interrupted or continued at a lower power if the optical detection signal indicates that the skin is irritated due to a too large dose of photon radiation.

In another embodiment the skin radiation device is designed for use in direct contact with the skin. In said embodiment the skin radiation device may have a contact sensor that only enables operation of the device when it is in contact with the skin.

The skin radiation apparatus may further comprise a memory for storing preset values. The preset values may comprise a preset value for the maximum and the minimum power density, for a frequency with which the primary radiation is modulated, a particular radiation wavelength range etc.

The memory may store more than one set of preset values for different users. The preset values may be initialized at a default value.

The skin radiation apparatus may include a mechanical massage facility. The mechanical massage facility may apply a mechanical massage in combination to the massage provided by the modulated primary radiation.

According to a second aspect of the invention a method is provided for providing a person's skin in a radiation area with photon radiation, having a total power density in the wavelength ranges from 300 to 700 nm, from 1900 to 2000 nm and from 2400 to 10.000 nm that is modulated between a first and a second mutually different value with a frequency of at least 0.1 Hz and of at most 10 Hz, wherein the first value is at least 20 mW/cm² and the second value is at most one fourth of the first value.

According to a third aspect of the invention a photon radiation power profile is provided for application at a person's skin in a radiation area, the profile having a total power density in the wavelength ranges from 300 to 700 nm, from 1900 to 2000 nm and from 2400 to 10.000 nm that is modulated between a first and a second mutually different value with a frequency of at least 0.1 Hz and of at most 10 Hz, wherein the first value is at least 20 mW/cm² and the second value is at most one fourth of the first value.

### BRIEF DESCRIPTION OF THE DRAWINGS

These and other aspects are described in more detail with reference to the drawing. Therein:

FIG. 1A shows a cross-section of the human skin,

FIG. 1B shows the penetration depth of photon radiation in the human skin as a function of the wavelength,

FIG. 1C shows the absorption of photon radiation for various substances present in the human skin as a function of the wavelength,

FIG. 1D shows the reflectivity of the human skin for photon radiation as a function of the wavelength,

FIG. 2 schematically shows an embodiment of a radiation apparatus according to the present invention,

FIG. 3 shows a part of the embodiment of FIG. 2 in more detail,

FIG. 4 shows a further embodiment of a radiation apparatus according to the present invention,

FIG. 5 shows for an embodiment of the present invention a relation between the current applied to the photon radiation source, the power density provided by the photon radiation source and the sensory perception by a test person, for a given area,

FIG. 6 shows for another embodiment of the present invention a relation between a percentage of test persons that sensed a temporal discontinuity of the applied photon radiation as a function of the off-time between subsequent photon radiation pulses.

FIG. 7A shows a first example of a photon radiation power profile for application at a person's skin,

FIG. 7B shows a second example of a photon radiation power profile for application at a person's skin,

FIG. 7C shows a third example of a photon radiation power profile for application at a person's skin,

FIG. 7D shows a fourth example of a photon radiation power profile for application at a person's skin.

### DETAILED DESCRIPTION OF EMBODIMENTS

In the following detailed description numerous specific details are set forth in order to provide a thorough understanding of the present invention. However, it will be understood by one skilled in the art that the present invention may be practiced without these specific details. In other instances, well known methods, procedures, and components have not been described in detail so as not to obscure aspects of the present invention.

The invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity.

It will be understood that, although the terms first, second, third etc. may be used herein to describe various elements, components, and/or sections, these elements, components, and/or sections should not be limited by these terms. These terms are only used to distinguish one element, component and/or section from another element, component, and/or section. Thus, a first element, component, and/or section discussed below could be termed a second element, component, and/or section without departing from the teachings of the present invention.

Unless otherwise defined, all terms (including technical and scientific terms) used herein have the same meaning as commonly understood by one of ordinary skilled in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein. All publications, patent applications, patents, and other references mentioned herein are incorporated by reference in their entirety. In case of conflict, the present specification, including definitions, will control. In addition, the materials, methods, and examples are illustrative only and not intended to be limiting.

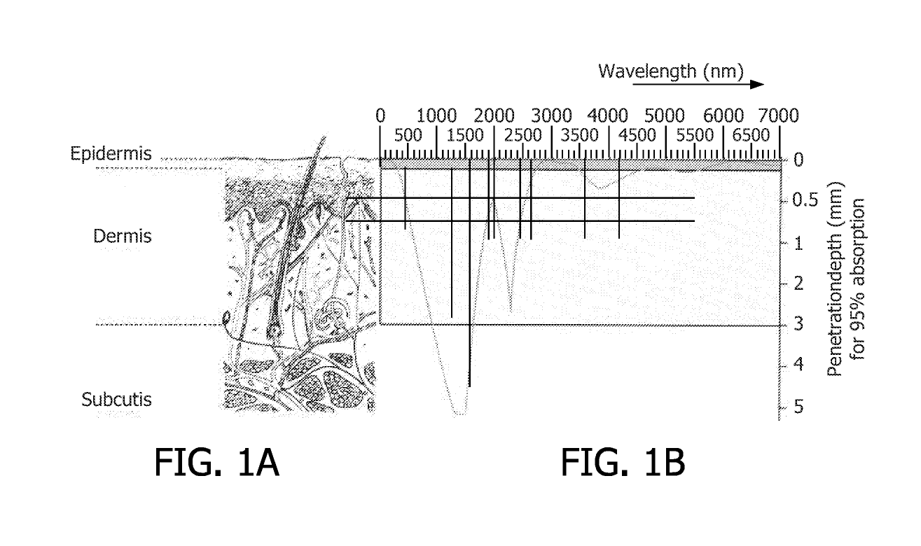

FIG. 1A schematically shows a cross-section of the human skin. The skin is composed of three functional layers: the epidermis, dermis and hypodermis or subcutis; each with its own unique functions.

The epidermis is the uppermost functional layer, usually comprised of 15-20 cell layers. The epidermis continually undergoes the birth, life and death of cells which are created at the base of the epidermis and, after a two-week migration, are shed at the surface.

The dermis, the next functional layer, is made up of cells, which produce fibers (collagen and elastin), and houses the elastic support of the skin. Nerve endings located in the dermis may detect changes in temperature and others may detect itch, pain etc. In particular receptors for sensing warmth are present in this functional layer at a depth of about 0.3 to 0.6 mm from the surface of the skin.

Finally the subcutis functions as a cushion and as a storage site for reserve energy for the body.

FIG. 1B shows the penetration depth of photon radiation as a function of the wavelength of the photon radiation. The penetration depth is defined as the depth where 95% of the impingent photon radiation is absorbed. In a vertical direction, indicating the depth of the skin, FIGS. 1A and 1B are at the same scale.

The penetration depth is mainly determined by the absorption of the photon radiation by the substances melanin, water and oxyhemoglobin, and by scattering within the skin layers. FIG. 1C shows the absorption in these substances as a function of the wavelength in a range from 300 to 2000 nm. Dependent on the wavelength range, light absorption in the skin is mainly caused by melanin, hemoglobin and water. Melanin is a pigment produced by the melanocytes, cells which are present in the epidermis and in the hairs, which extend outside from the dermis. Hemoglobin is present in the blood in the blood vessels especially in the dermis. Water is substantially present in each of the functional layers of the skin. Generally speaking photon radiation in the UVB range is substantially absorbed by melanin in the epidermis, so that it doesn't penetrate much into the dermis. UVA radiation, penetrates a bit also in the dermis, and blue radiation penetrates slightly deeper into the dermis than UVA radiation. At longer wavelengths the penetration depth increases to a penetration depth of about 5 mm at 1300 nm, because the absorption of melanin and hemoglobin decreases. At wavelengths above 1500 nm however the absorption of water increases to a substantial value, therewith contributing to a decrease of the penetration depth for those wavelengths. This explains a strong decrease of the penetration depth to about 0.5 mm for a wavelength of 1950 nm. The penetration dept has a second maximum of 3 mm at a wavelength of 2300 nm and a second minimum of about 0 mm for 2850 nm. Longer wavelengths in the range from 2850 to 10.000 nm superficially penetrate the skin.

FIG. 1D shows the reflectivity of the skin as a function of wavelength. In a wavelength range of about 300 to 1500 nm the reflectivity is relatively high. For a dark skin the amount of reflection raises from about 10% to a maximum of about 45% at a wavelength of 1000 nm and decreases to about 10% for a wavelength of 1400 nm and higher. For a white skin the amount of reflection increases from about 10% at 300 nm to a maximum of about 70% at a wavelength of 700 nm and decreases again to about 10% for wavelengths of 1400 nm and higher.

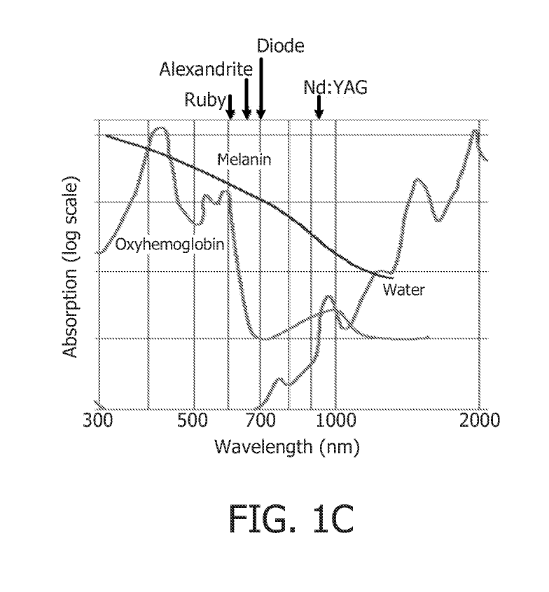

FIG. 2 shows a skin radiation apparatus 10 for providing a person's skin 20 near a radiation area 12 of the skin radiation apparatus with modulated photon radiation 14. The apparatus 10 comprises a photon radiation source 18 for generating the photon radiation 14 and a modulation facility 16. During operation the modulation facility causing a modulation of the total power density of photon radiation in the wavelength ranges from 300 to 700 nm, from 1900 to 2000 nm and from 2400 to 10.000 nm in the radiation area 12 between first and a second value. The total power density across said ranges is modulated with a frequency of at least 0.1 Hz and of at most 10 Hz. In the embodiment shown the first value is at least 20 mW/cm² and the second value is at most one fourth of the first value.

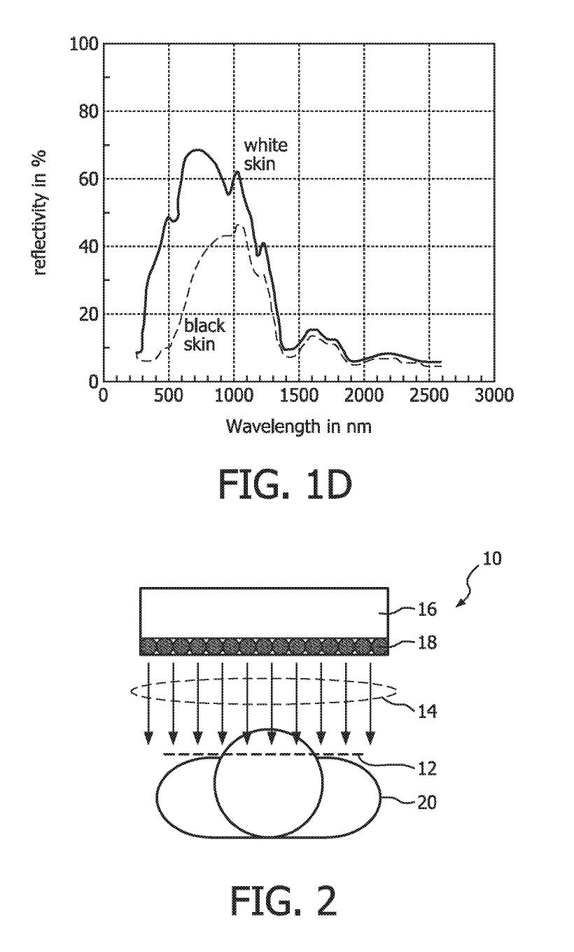

FIG. 3 shows the modulation facility 16 in more detail. The modulation facility 16 comprises a modulated power supply facility 161 that provides the photon radiation source 18 with a modulated supply power. In this case the photon radiation source 18 comprises one or more LED's and the modulated supply power is provided in the form of a modulated supply current. In this embodiment the power density is modulated simultaneously in the entire radiation area.

In the embodiment shown the modulated power supply facility 161 comprises a timer 162 for interrupting operation of the apparatus after a predetermined time. The predetermined time may be set via a user interface 163. The preset time may be stored in a memory 164. Furthermore the modulation facility 16 comprises a distance sensing facility 165 for generating a distance signal indicative for a distance between the photon radiation source 18 and the radiation area 12 and an optical detection facility 166 for providing an optical detection signal.

In another embodiment the skin radiation apparatus may be applied in contact with the skin. In that embodiment the skin radiation apparatus may have a contact sensor that disables operation of the skin radiation apparatus if it is not in contact with the skin.

In an exemplary embodiment of the skin radiation apparatus the photon radiation source is formed by a plurality, here 3, of InGaN LEDs of type Luxeon Blue, manufactured by Philips Lumileds. These LED's provide photon radiation with a wavelength of 420 nm. The power density of the photon radiation in the radiation area could be varied from 0 to about 200 mW/cm² by varying the supply current in a range from 0 to 800 mA.

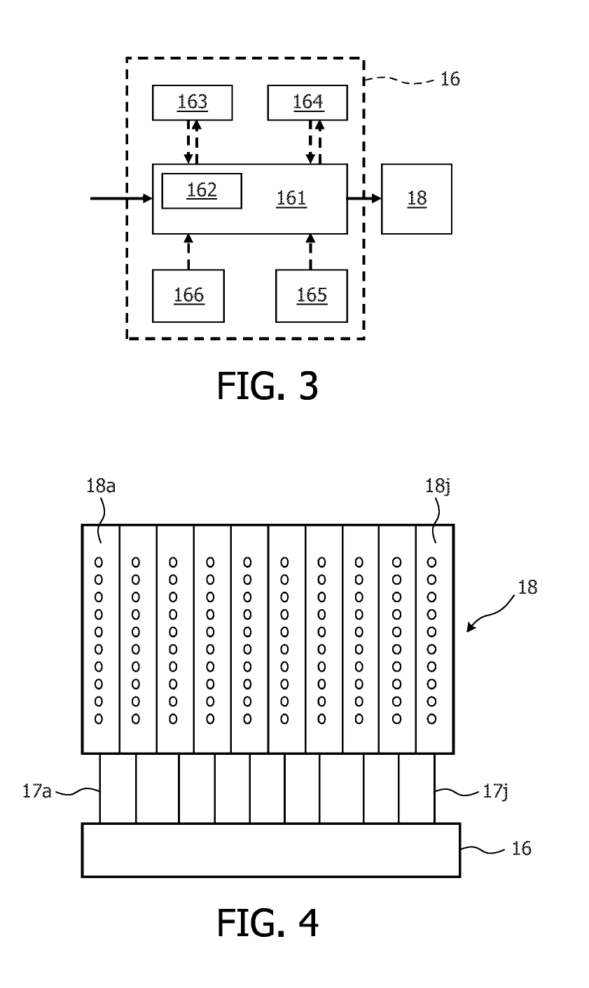

FIG. 4 shows a further embodiment of a radiation apparatus according to the present invention. Therein the radiation source 18 comprises a plurality of radiation modules 18a, . . . , 18j that are coupled by respective supply lines 17a, . . . , 17j to a power supply 16 and that are switched on during mutually different time intervals (means for switching not shown). Alternatively each radiation module may have its own power supply, and a central controller activates the respective power supplies during mutually different time intervals. In the embodiment shown the radiation source 18 comprises 10 radiation modules 18a, . . . , 18j that each irradiate the skin in a respective sub-area of the radiation area. In this embodiment the radiation modules 18a, . . . , 18j are formed by parallel strips, each comprising a plurality, 10 in this case, of LED's. The radiation source 18 may have a mode of operation wherein each radiation module is switched on when its predecessor is switched off. When the last radiation module 18j in the sequence is switched off the first radiation module 18a is switched on again. Instead of switching on a strip at the moment that its predecessor is switched off, the time intervals during which the radiation module are switched on may overlap. Alternatively some time may lapse between the point in time that a radiation module is switched off and the point in time that a next radiation module is switched on.

It is not necessary that each radiation module 18a, . . . , 18j is driven with the same supply power. For example in a first cycle each consecutive radiation module may be powered at a higher level so that it provides the skin with a higher power density than its predecessor. In a second cycle, following the first cycle each consecutive radiation module may be driven with a lower power. This pattern may be repeated.

In an exemplary embodiment the modulation facility 16 is a current source capable of providing a current in a range corresponding with said power density range that is alternately switched on and off with a frequency that is controllable in a range of 0.01 and 100 Hz.

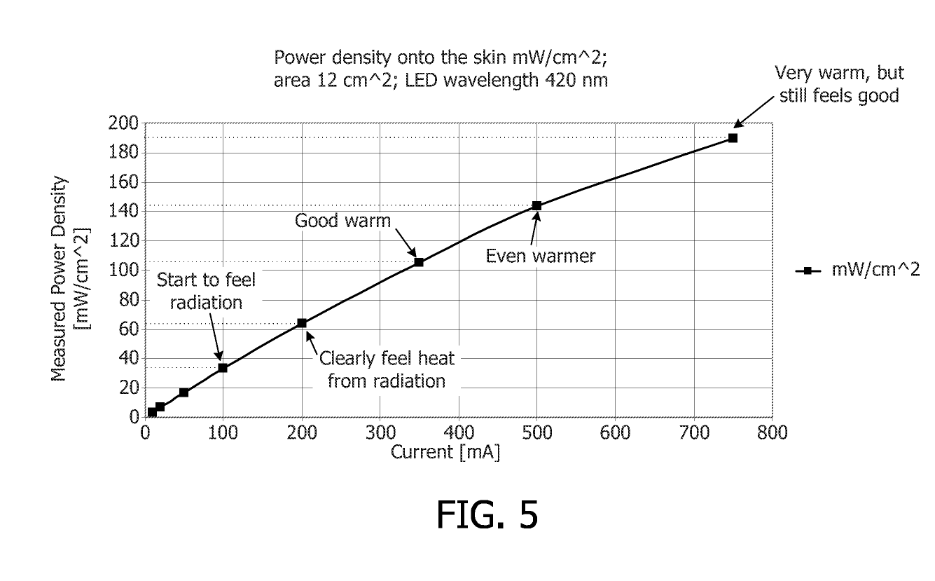

The apparatus was tested with a person having a light skin, type 2. The radiation area was about 20 cm². The results are shown in FIG. 5. The graph therein shows the measured power density in the radiation area 12 as a function of the supply current I. FIG. 5 further indicates the sensatory experience of the test person for concrete settings of the power density (the rectangular dots in the graph). At a power density of 19 mW/cm² the test person did not yet sense warmth. At a power densities of 35 mW/cm² and higher the photon radiation was sensed. At a power density of 190 mW/cm² the photon radiation was sensed as very warm, but still comfortable. It is assumed that power densities higher than 190 mW/cm² are also acceptable, taking into account that in an apparatus according to the invention the photon radiation is provided in a pulsed fashion, so that that the skin is allowed to cool between subsequent photon radiation pulses.

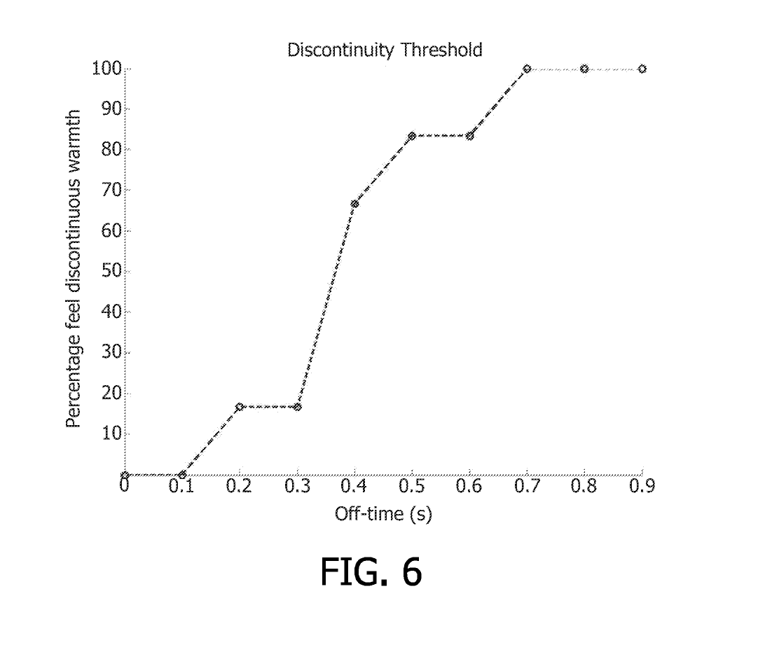

The effect of the time period between subsequent pulses was investigated in a further experiment. In this experiment a broad spectrum lamp of type Reflect IR-P1N manufactured by ICX photonics was used to apply photon radiation pulses with a power density of 263 mW/cm². The photon radiation pulses, having a duration of 2 s, were applied to an area of 22 cm² of the skin, type 2, of 6 test persons. The separation between subsequent pulses was varied between 0.0 to 1.0 s. The results are presented in FIG. 6. At a separation of 0.1 s none of the test persons perceived a discontinuity in the photon radiation provided by the photon radiation source. At a separation of 0.2 s one test person already sensed the absence of photon radiation between subsequent photon radiation pulses. At a separation of 0.4 s a sudden increase is observed of the number of test persons that perceived the temporal discontinuity of the photon radiation. At a separation of 0.7 s and higher all test persons felt a discontinuity in the photon radiation.

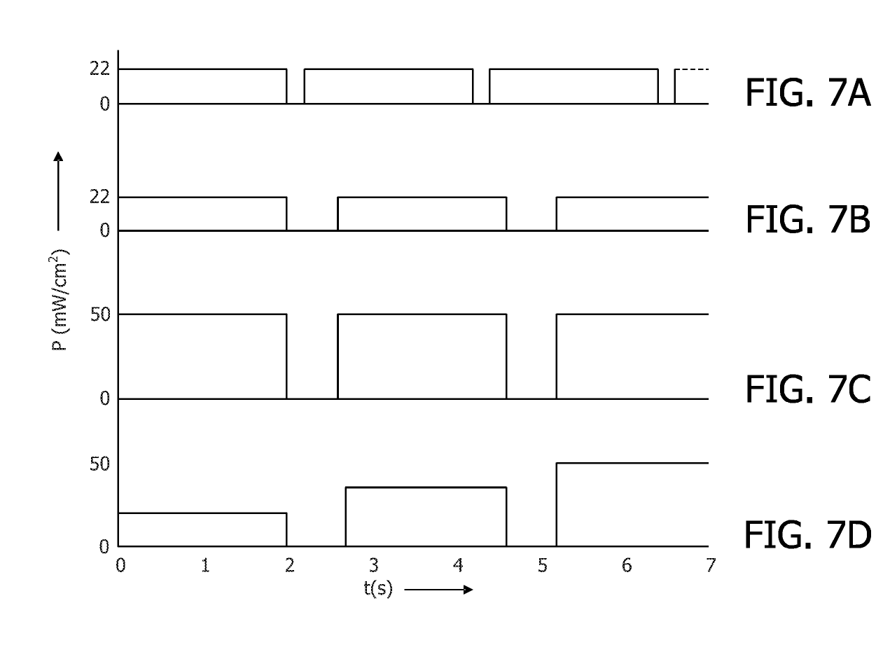

FIGS. 7A to 7D shows several examples of photon radiation power profiles according to the present invention.

The vertical axis indicates the total power density (in mW/cm²) of photon radiation in the wavelength ranges from 300 to 700 nm, from 1900 to 2000 nm and from 2400 to 10.000 nm. The horizontal axis indicates the time in seconds. In the example shown in FIG. 7A, the total power density in said wavelength ranges is modulated pulsewise between a first value of 22 mW/cm² and a second value of 0 mW/cm². The pulse duration is 2 s and the time interval between subsequent pulses is 0.2 s.

In the example shown in FIG. 7B, the total power density in said wavelength ranges is modulated pulsewise between a first value of 22 mW/cm² and a second value of 0 mW/cm². Also in this case the pulse duration is 2 s, however the time interval between subsequent pulses is 0.6 s.

In the example shown in FIG. 7C, the total power density in said wavelength ranges is modulated pulsewise between a first value of 50 mW/cm² and a second value of 0 mW/cm². The pulse duration is 2 s, and the time interval between subsequent pulses is 0.6 s.

The example shown in FIG. 7D differs from the previous examples in that the total power density is gradually incremented with each subsequent pulse. In this example the first pulse has a power density with a first value of 20 mW/cm², the second pulse has a power density with a first value of 35 mW/cm² and the third pulse has a power density with a first value of 50 mW/cm². The pulse duration is 2 s, and the time interval between subsequent pulses is 0.6 s. In practice the power density of the pulses may be increased more gradually. For example in a sequence of 100 pulses the power density of the pulse, i.e. the first level may be gradually increased from 20 mW/cm² for the first pulse to 60 mW/cm² for the last pulse in the sequence.

In the examples presented in FIG. 7, the total power density was switches between a first level, e.g. 22 mW/cm², and a second level of 0 mW/cm². However, the massage effect may also be achieved without shutting off the total power density but by switching the total power density to a second value substantially lower that the first value such that a difference in warmth sensation is felt. This difference in warmth sensation is thought to be felt if the second value is at most one fourth of the first value.

Dependent on the skin type, the skin condition, the sensitivity and personal preferences of the person, one of these or other photon radiation power profiles according to the present invention may be selected when carrying out a method for providing a person's skin in a radiation area with photon radiation.

In the claims the word “comprising” does not exclude other elements or steps, and the indefinite article “a” or “an” does not exclude a plurality. A single component or other unit may fulfill the functions of several items recited in the claims.

The mere fact that certain measures are recited in mutually different claims does not indicate that a combination of these measures cannot be used to advantage. Any reference signs in the claims should not be construed as limiting the scope. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

1. Skin radiation apparatus for providing a person's skin in a radiation area of the apparatus with modulated photon radiation, the apparatus comprising a photon radiation source for generating the photon radiation and a modulation facility for causing a modulation of at least the total power density of photon radiation in the wavelength ranges from 300 to 700 nm, from 1900 to 2000 nm and from 2400 to 10.000 nm in at least a sub-area of the radiation area, between a first and a second mutually different value, with a frequency of at least 0.1 Hz and of at most 10 Hz, wherein the first value is at least 20 mW/cm² and the second value is at most one fourth of the first value.

2. Skin radiation apparatus according to claim 1, wherein the photon radiation has a wavelength in one or more of the ranges from 1900 to 2000 nm and from 2400 to 10.000 nm.

3. Skin radiation apparatus according to claim 1, wherein the photon radiation has a wavelength in one or more of the ranges from 300 to 500 nm, 1900 to 2000 nm, from 2400 to 2600 nm and from 3600 to 4200 nm.

4. Skin radiation apparatus according to claim 1, wherein the photon radiation has a wavelength in one or more of the ranges from 1900 to 2000 nm, from 2400 to 2600 nm and from 3600 to 4200 nm.

5. Skin radiation apparatus according to claim 1, wherein the first value of the power density is at least 50 mW/cm².

6. Skin radiation apparatus according to claim 1, wherein the first value of the power density is at most 200 mW/cm².

7. Skin radiation apparatus according to claim 1, wherein the modulation facility modulates a power supplied to the photon radiation source.

8. Skin radiation apparatus according to claim 1, comprising a facility for providing the radiation area with additional radiation.

9. Skin radiation apparatus according to claim 8, wherein the additional radiation has a wavelength in the range of 700 to 1600 nm.

10. Skin radiation apparatus according to claim 8, wherein the additional radiation is modulated synchronously with the modulation caused by the modulation facility.

11. Skin radiation apparatus according to claim 8, wherein the additional radiation is provided with a substantially constant power density.

12. Skin radiation apparatus according to claim 1, further comprising a distance sensing facility for generating a distance signal indicative for a distance between the photon radiation source and the radiation area.

13. Skin radiation apparatus according to claim 1, further comprising an optical detection facility for providing an optical detection signal.

14. Method for providing a person's skin in a radiation area with photon radiation, having a total power density in the wavelength ranges from 300 to 700 nm, from 1900 to 2000 nm and from 2400 to 10.000 nm that is modulated, in at least a sub-area of the radiation area, between a first and a second mutually different value with a frequency of at least 0.1 Hz and of at most 10 Hz, wherein the first value is at least 20 mW/cm² and the second value is at most one fourth of the first value.

15. Photon radiation power profile for application at a person's skin in a radiation area, the profile having a total power density in the wavelength ranges from 300 to 700 nm, from 1900 to 2000 nm and from 2400 to 10.000 nm that is modulated, in at least a sub-area of the radiation area, between a first and a second mutually different value with a frequency of at least 0.1 Hz and of at most 10 Hz, wherein the first value is at least 20 mW/cm² and the second value is at most one fourth of the first value.
